(19) United States
(12) Patent Application Publication
(10) Pub. No.: US 2026/0063535 A1
(43) Pub. Date: Mar. 5, 2026

(54) SYSTEM AND APPARATUS FOR MEASURING SCATTERED LIGHT SIGNALS FROM PRESSURIZED SOLUTIONS AND METHOD OF ASSEMBLY

(71) Applicant: UNIVERSITY OF DELAWARE, Newark, DE (US)

(72) Inventors: Jordan Evan Berger, Newark, DE (US); Christopher J. Roberts, Newark, DE (US); Wei Qi, Newark, DE (US); Susana Marujo Teixeira, Newark, DE (US); Christopher Sloey, Newark, DE (US)

(73) Assignee: University of Delaware, Newark, DE (US)

(21) Appl. No.: 19/101,051

(22) PCT Filed: Aug. 7, 2023
(86) PCT No.: PCT/US2023/029655

§ 371 Date: Feb. 4, 2025

Related U.S. Application Data
(60) Provisional application No. 63/395,364, filed on Aug. 5, 2022.
(60) Provisional application No. 63/506,616, filed on Jun. 7, 2023.

(51) Int. Cl. G01N 21/03; G01N 21/51; G01N 33/577

(52) U.S. Cl. CPC G01N 21/0332; G01N 21/51; G01N 33/577; G01N 2021/0389; G01N 2021/513

(57) ABSTRACT

A system for evaluating light scattering properties of a liquid sample includes a light scattering instrument with a chassis and a laser source that emits a laser beam along a path. An apparatus includes a pressure cell with a chamber for containing a fluid pressurized up to 350 MPa and a plurality of light-transmissive windows, including a beam entry window and a beam exit window positioned in the path of the laser beam, and one or more windows positioned orthogonal to the path of the laser beam. A biological sample is received in the pressure cell. A plurality of detectors includes a transmission detector disposed adjacent the beam exit window, or light scattering detectors positioned adjacent the windows orthogonal to the path of the laser beam and configured to detect light scattered from the laser beam passing through the sample. An external condition-inducing system provides a fluid temperature and pressure.

### CROSS-REFERENCE TO RELATED APPLICATIONS

This application claims priority from U.S. Provisional Application Ser. No. 63/395,364, titled “Apparatus for Measuring Scattered Light Signals from Pressurized Solutions,” filed Aug. 5, 2022, and U.S. Provisional Application Ser. No. 63/506,616, titled “Apparatus for Measuring Scattered Light Signals from Pressurized Solutions and Method of Assembly,” filed Jun. 7, 2023, the entireties of both of which are incorporated herein by reference.

### STATEMENT REGARDING FEDERALLY SPONSORED RESEARCH

This invention was made with government support under 70NANB20HI33 and 70NANB12H302 awarded by the National Institute of Standards and Technology, U.S. Department of Commerce. The government has certain rights in the invention.

### BACKGROUND OF THE INVENTION

Elevated hydrostatic pressure has increasingly become a variable used for characterizing partially unfolded intermediate states of proteins. Pressure may perturb protein structures as intermolecular interactions are particularly pressure-sensitive. The effects of pressure are biomolecule specific, and protein molecular structure is altered by pressure on length scales that range from angstrom (atomistic) to nanometer (quaternary structure), with measurable effects from individual amino acids, to domains or full quaternary structure. In certain pressure conditions, there is no direct effect on covalent bonds. Instead, pressure acts to stabilize protein structures with comparatively lower partial specific volumes in solution, achieved by compaction and/or partial unfolding relative to the higher partial specific molar volume folded state. Pressure effects on reversible intermolecular protein-protein interactions (PPIs) are not as well characterized because they can only be measured through in situ techniques.

Light scattering techniques have been widely employed to study colloidal, conformational, and hydrodynamic properties of protein molecules at ambient pressure. In addition, there is an increasingly dominant role of temperature sensitive Monoclonal Antibodies (MAbs) in the current pharmaceutical market. There is also a need to understand biomolecular adaptations to extreme conditions in a broader context. Light scattering techniques allows for investigation of the shape, size, and interactions of MAb molecules/aggregates as a function of pressure and temperature. Conventional methods use MAb aggregation at high temperatures to extrapolate comparatively slow effects at typical storage conditions (refrigerated/freezing). However, the processes involved in varying temperatures (e.g. high vs low temperature) concern different biomolecular phases of the energetic landscape. Additionally, despite their ability to trigger permanent aggregation, transient biomolecular states are very short lived and therefore difficult to study. Pressure is known to stabilize such structures, enabling their study and a deeper understating of the mechanisms involved. High pressure also enables access to low temperature solution studies in the absence of ice, allowing for light scattering data to be collected and more accurate prediction of long-term storage stability.

The use of pressure cells on light-scattering equipment has so far been limited to high pressure gas environments. Conventional techniques do not readily provide single molecule structural information for solutions under high pressure and isolated from the pressurizing medium. Typical nuclear magnetic resonance (NMR) techniques are limited in molecule size and assignment of resonances for a typical MAb require significant efforts in terms of time, expensive equipment, and man-power. Scattering techniques can tackle large molecules using intense beams located at synchrotron or neutron research centers, but access to such large-scale facilities remains limited.

Thus, it is of interest to develop improvements in a high-pressure light scattering apparatus equipped with low temperature capabilities and methods of assembly thereof.

### SUMMARY OF THE INVENTION

One aspect of the invention is a system for evaluating light scattering properties of a liquid sample. The system includes a light scattering instrument comprising a chassis and a laser source configured to emit a laser beam along a path. The system also has an apparatus configured to be secured to the light scattering instrument. The apparatus includes a pressure cell mounted to the chassis. The pressure cell includes a chamber defined by one or more walls and configured to contain a fluid pressurized up to 350 MPa. The pressure cell also has a plurality of light-transmissive windows in the one or more walls, including at least a beam entry window and a beam exit window positioned in the path of the laser beam, and one or more windows positioned orthogonal to the path of the laser beam, and a sample enclosure positioned in the path of the laser beam, the sample enclosure isolated from, but in temperature and pressure communication with the fluid. The system also comprises an external condition-inducing system configured to provide a predetermined temperature and pressure of the fluid within the pressure cell chamber. A biological sample is received in the sample enclosure of the pressure cell. The system also includes a plurality of detectors positioned adjacent one or more of the plurality of windows. The plurality of detectors includes one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample.

Another aspect of the invention is an apparatus configured to be secured to a light scattering instrument having a chassis and a laser source configured to emit a laser beam along a path. The apparatus includes a pressure cell mounted to the chassis. The pressure cell has a chamber defined by one or more walls and configured to contain a fluid pressurized up to 350 MPa. The pressure cell also includes a plurality of light-transmissive windows in the one or more walls, including at least a beam entry window and a beam exit window positioned in the path of the laser beam, and one or more windows positioned orthogonal to the path of the laser beam. A sample enclosure is positioned in the path of the laser beam, such that the sample enclosure is isolated from, but in temperature and pressure communication with the fluid. The apparatus has an external condition-inducing system configured to provide a predetermined temperature and pressure of the fluid within the pressure cell chamber. A biological sample is received in the sample enclosure of the pressure cell. The apparatus includes a plurality of detectors positioned adjacent one or more of the plurality of windows. The plurality of detectors has one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample.

Still another aspect of the invention is a method for retrofitting an atmospheric light scattering instrument, the atmospheric-pressure light scattering instrument having a chassis and a laser source configured to emit a laser beam along a path. The method includes a step of mounting a pressure cell to the chassis. The pressure cell has a chamber defined by one or more walls and configured to contain a fluid pressurized up to 350 MPa. The pressure cell also includes a plurality of light-transmissive windows in the one or more walls, including at least a beam entry window and a beam exit window positioned in the path of the laser beam, and one or more windows positioned orthogonal to the path of the laser beam. A sample enclosure is positioned in the path of the laser beam, such that the sample enclosure isolated from, but in temperature and pressure communication with the fluid. The method includes a step of providing an external condition-inducing system configured to provide a predetermined temperature and pressure of the fluid within the pressure cell chamber. The method also has a step of providing one or more openings in the chassis for facilitating a connection between the pressure cell and the external condition-inducing system. The method includes inserting a biological sample into the sample enclosure of the pressure cell. The method also has a step of positioning a plurality of detectors adjacent the pressure cell. The plurality of detectors has one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample. The method comprises steps of connecting a pressure cable of the external condition-inducing system to the pressure cell via a pressure inlet; emitting a laser beam along a path toward to the pressure cell, such that when the laser beam enters the sample enclosure of the pressure cell, the light scattered by the biological sample is transmitted through the plurality of windows; and detecting one or more characteristics of said scattered light by the plurality of detectors.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. This emphasizes that according to common practice, the various features of the drawings are not drawn to scale unless otherwise indicated. On the contrary, the dimensions of the various features may be expanded or reduced for clarity. Included in the drawings are the following figures:

FIG. 1A depicts a schematic of a system of evaluating light scattering properties of a liquid sample in accordance with an exemplary embodiment of the invention;

FIG. 1B depicts an image of an exemplary pressure cell of the system of FIG. 1A;

FIG. 1C depicts a schematic of an exemplary apparatus of the system of FIG. 1A;

FIG. 1D depicts a schematic of exemplary light scattering properties evaluated by the system of FIG. 1A;

FIGS. 2A-2B depict graphs illustrating exemplary characteristics of the scattered light as expressed by calculated and plotted excess Rayleigh ratio profiles for certain concentrations of monoclonal antibodies in accordance with tests performed using the system of FIG. 1A;

FIGS. 3A-3B depict graphs illustrating exemplary characteristics of the scattered light as expressed by calculated and plotted excess Rayleigh ratio profiles as a function of time in accordance with tests performed using the system of FIG. 1A;

FIG. 4 depicts a graph illustrating exemplary characteristics of the scattered light as expressed by monomer loss profiles in accordance with tests performed using the system of FIG. 1A;

FIGS. 5A-5E depict graphs illustrating exemplary characteristics of the scattered light as expressed by excess Rayleigh ratio profiles in accordance with tests performed using the system of FIG. 1A;

FIG. 6 depicts a graph illustrating exemplary characteristics of the scattered light as expressed by monomer loss profiles from pressure cycle incubations in accordance with tests performed using the system of FIG. 1A;

FIGS. 7A-7B depict graphs illustrating exemplary characteristics of the scattered light as expressed by excess Rayleigh ratio profiles for certain concentrations of lysozyme in accordance with tests performed using the system of FIG. 1A; and

FIG. 8 depicts a schematic of a method of assembling an apparatus of the system of FIG. 1A in accordance with an exemplary embodiment of the invention;

### DETAILED DESCRIPTION OF THE INVENTION

Aspects of this invention relate to methods, apparatuses, and systems for evaluating light scattering properties of a liquid sample.

Although the invention is illustrated and described herein with reference to specific embodiments, the invention is not intended to be limited to the details shown. Rather, various modifications may be made in the details within the scope and range of equivalents of the claims and without departing from the invention.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

While the exemplary embodiments of the invention are described herein with respect to lysozymes or monoclonal antibodies (MAbs), it will be understood that the invention is not so limited. Suitable applications for systems, apparatuses, and/or methods of the present invention include, for example, works studying underlying molecular aggregation mechanisms, static light scattering studies to investigate protein-protein interactions at elevated pressure as well as the link between protein aggregation and PPIs at high pressure (HP) and atmospheric conditions, and studies to assess intermediate states of other molecules, such as viruses (or virus-like particles), nucleic acid-based therapeutics, and other multimeric proteins. Other suitable applications will be readily understood by one of ordinary skill in the art from the description herein.

As used herein and throughout the specification, the term “biological sample” is intended to encompass any type of biological specimen, and is not limited to a specific type, number, or configuration of structural components of living organisms (e.g. protein, virus, etc.).

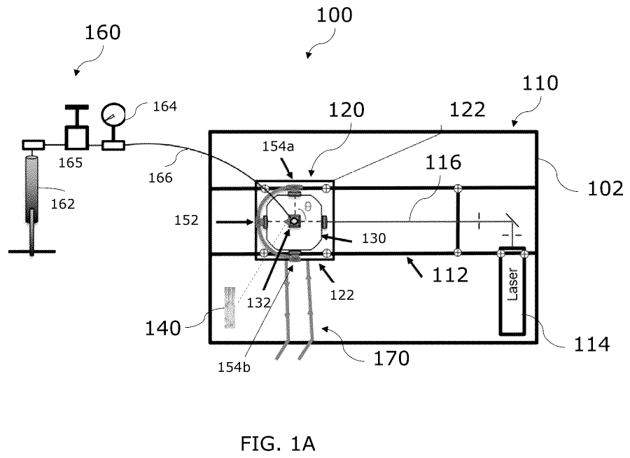

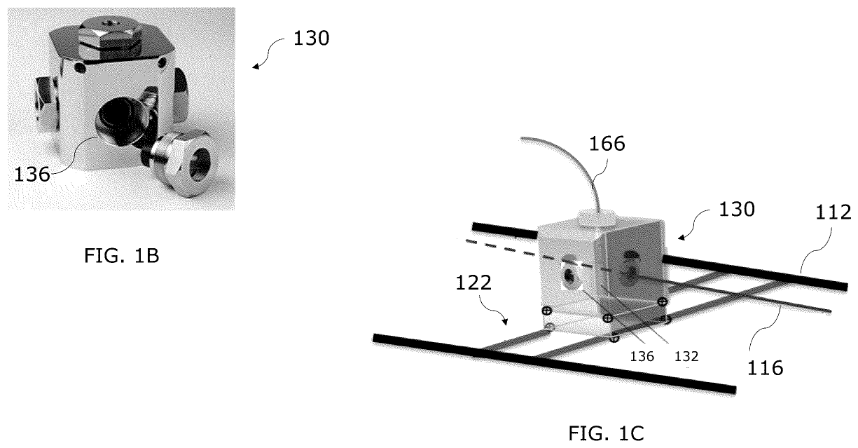

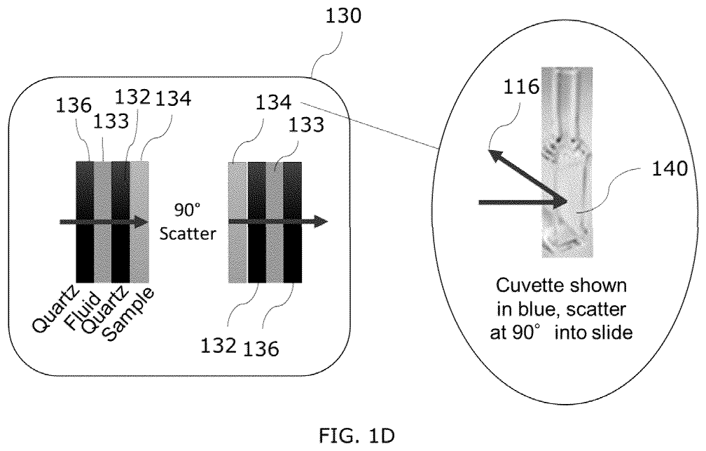

Referring generally to FIGS. 1A-1D, a system 100 for evaluating light scattering properties of a liquid sample is disclosed. Generally, system 100 includes a light scattering instrument 110, such as an atmospheric-pressure light scattering instrument, an apparatus 120 configured to be secured to the light scattering instrument 110, a pressure cell 130, a biological sample 140 received within the pressure cell 130, and a plurality of detectors 150. As will be discussed in the Example below, in situ aggregation behavior of biological samples 140, such as MAbs, demonstrate the function and physical parameters of the system 100 having apparatus 120. For example, the extent of aggregation during isothermal and isobaric processes, as well as the effects of pressure cycling and total ionic strength can be studied using the inventive system 100. Although the results in the Example are discussed in the specific context of conformational stability and long-term (up to three weeks) high pressure incubations, as well as in the context of pressure effects on PPIs of lysozyme, the general use of elevated pressure for perturbing protein-protein interactions or influencing reversible and irreversible protein aggregation or self-association, can be studied with system 100. Additional details of system 100 are discussed below.

In an exemplary embodiment, the light scattering instrument 110 includes a chassis 112 and a laser source 114 configured to emit a laser beam along a path. In one example, the laser source 114 comprises a laser having a wavelength of 658 nm and a power output of 50 mW. One skilled in the art would understand from the description herein that the system 100 is not limited to a type or model of light scattering instrument 110. Instead, the apparatus 120 is configured to be customizable and usable with a variety of light scattering instruments 110. In one example, when the light scattering instrument 110 comprises an atmospheric-pressure light scattering instrument, apparatus 120 comprises an adapter 122 configured to be secured to the chassis 112. In one example, as shown in FIG. 1C the adapter includes a plurality of openings having a complementary configuration or geometry relative to an attachment surface of a pressure cell 130. For example, the attachment surface of the pressure cell 130 is configured to be received by the plurality of openings of the adapter 122. Additionally, or optionally, the adapter 122 is movable or translatable along an axis of the chassis 112 for alignment, such that when the adapter 122 and the pressure cell 130 mounted thereon is properly calibrated and aligned, the entry of a laser beam 124 from the laser source 114 into the pressure cell 130 is permitted or facilitated.

1. In an exemplary embodiment, the pressure cell 130 is mounted to the chassis 112 includes a sample enclosure 132 comprising a cuvette 134. In one example, the cuvette 134 comprises a square bottom quartz cuvette. The sample enclosure 132 comprises a pressure-transmitting closure, such as a stopper, disposed between the fluid in the chamber and the sample 140 in the sample enclosure 132, the pressure-transmitting closure configured to reversibly change in configuration to cause a change in pressure of the fluid to be translated into a proportional change in pressure of the sample 140. In one example, secured to the cuvette 134 is a stopper configured to translate along a neck of the cuvette 134 to transmit pressure to the biological sample from the external condition-inducing system 160/170 (discussed below). The invention is not limited to any particular configuration of or type of sample enclosure, nor any particular configuration of the pressure-transmitting closure of the sample enclosure. For example, a closure comprising a sufficiently flexible and secure membrane separating the fluid from the sample may also respond suitably to changes in pressure. It is preferable, however, that the path of the laser beam through the sample enclosure, as well as the path of scattered light to the scattered light detector(s), is undisturbed by the changes in the configuration of the closure. In one example, the sample enclosure 132 is configured to receive a biological sample 140 comprising proteins. The biological sample 140 may comprise proteins or monoclonal antibodies, for example. The pressure cell 130 also has a chamber defined by one or more walls and configured to contain a fluid pressurized up to 350 MPa. The pressure cell 130 includes a plurality of windows 136, such as a plurality of light-transmissive windows in the one or more walls. In an exemplary embodiment, the plurality of windows 136 has at least a beam entry window and a beam exit window positioned in the path of the laser beam 116 and one or more windows 136 positioned orthogonal to the path of the laser beam 116. In this way, the sample enclosure 132 is positioned in the path of the laser beam 116, such that the sample enclosure is isolated from, but in temperature and pressure communication with the fluid in the chamber of the pressure cell 130. In one example, the plurality of windows 136 comprises quartz.

Still further, the pressure cell 130 is configured to contain a hydrostatic fluid 133 (e.g. water, ethanol) surrounding (or at least in thermal and pressure communication with) the sample. The pressure cell 130 also may include means for controlling the temperature thereof. For example, the pressure cell 130 may have a built in path (not shown) for circulating a temperature control liquid (e.g. water, ethanol) supplied via tubes 170 from a controlled liquid temperature source (e.g. an external bath circulator (not shown)), attached to the tubes. A cell constructed of materials with suitable high thermal conductivity (e.g. stainless steel) will rapidly stabilize the temperature of the cell and the hydrostatic fluid contained therein. In this way, the fluid in the chamber may be stabilized at a set pressure with temperature separately controlled by a temperature control system to transmit heat or cold and pressure from the fluid therein to the biological sample 140 secured within the pressure cell 130. In other embodiments, the fluid 133 in the chamber may circulate through the chamber at a set pressure and may be directly temperature controlled with heat exchangers of any configuration known in the art. In one example, the fluid comprises a polyethylene glycol-water mixture capable of reaching a temperature range between −20° C. and 90° C. Other materials for use as promoting heat transfer would be known to one of ordinary skill in the art. To facilitate this, external condition-inducing system 160 includes one or more tubes 170 configured to facilitate a fluid connection between an external bath circulator (not shown) and the temperature control path within the pressure cell, which controls temperature of the pressurized fluid.

In an exemplary embodiment, system 100 further includes an external condition-inducing system 160/170 configured to provide a predetermined condition. Thus, the external condition-inducing system is configured to create an environment having the predetermined condition for the biological sample 140. In one example, the external condition-inducing system 160/170 is configured to provide and/or maintain a predetermined temperature and pressure of the fluid within the chamber of the pressure cell 130, and in turn, the biological sample 140 secured within the pressure cell 130. In one example, the temperature is in a range between −20° C. and 90° C. In another example, predetermined condition comprises temperature in a range between −15° C. and 60° C.

Additionally, or optionally, the predetermined condition includes a pressure applied to the fluid within the pressure cell 130. To provide an applied pressure, the external condition-inducing system 160/170, or more particularly, external pressure-inducing system 160 comprises a pressure source, such as a pump (or compressor or reservoir of compressed fluid) 162 connected to a pressure inlet of the pressure cell 130. The external pressure-inducing system 160 also includes a pressure gauge 164 positioned between the pump 162 and the pressure cell 130, and the pressure gauge 164 is configured to measure applied pressure to pressure cell 130. A valve 165 may optionally be provided between the pump and the gauge to hold pressure once a desired pressure is achieved using the pump. In other embodiments, the valve may be used for controlling or cycling pressure between the pressure source and atmospheric pressure (or a source of vacuum) to provide a desired set pressure. In an exemplary embodiment, the predetermined condition comprises the pressure in a range between ambient pressure to 350 MPa. In particular, the predetermined condition comprises the pressure in a range between 0.1 MPa and 350 MPa.

In operation, the plurality of detectors 150 are positionable adjacent the pressure cell 130. In an exemplary embodiment, a plurality of detectors 4150 are positioned adjacent one or more of the plurality of windows 136. Additionally or optionally, the plurality of detectors 150 has one or more of a transmission detector 152 disposed adjacent the beam exit window, and one or more light scattering detectors 154a/154b positioned adjacent the one or more windows 136 orthogonal to the path of the laser beam 116 and configured to detect one or more characteristics of light scattered from the laser beam 116 passing through the sample 140. For example, the plurality of light-transmissive windows 136 includes two orthogonal windows, and light scattering detectors 154a/154b positioned adjacent said two orthogonal windows 136. In another example, the plurality of light-transmissive windows 136 includes two orthogonal windows, wherein the light scattering detector comprises an SLS 154a and a DLS 154b positioned adjacent orthogonal windows 136 and opposite one another. In another exemplary embodiment, the plurality of detectors includes a transmission detector 152 and a dynamic light scattering detector (DLS) 154b and/or a static light scattering detector (SLS) 154a. The DLS and/or SLS are positionable on opposite surfaces of the pressure cell 130 and along an orthogonal orientation relative to the transmission detector 152.

In one example, the SLS 154a comprises a silicon PIN photodiode. In this configuration, the laser source 114 is arranged to emit a laser beam 116 along a path toward to the pressure cell 130, such that when the laser beam 116 enters the sample enclosure 132 of the pressure cell 130, the light scattered by the biological sample 140 is transmitted through the plurality of windows 136, and one or more characteristics of said scattered light is detected by the plurality of detectors 150. Although depicted in FIG. 1A as including both SLS 154a and DLS 154b in opposite windows, embodiments may have both the SLS and DLS detectors but positioned in different windows than those shown (including in windows that are not opposite one another, but both still orthogonal to the laser beam path), one of the SLS or the DLS detector positioned in any one of the windows orthogonal to the laser beam path, two (or more) SLS detectors, or two (or more) DLS detectors.

Although depicted as having a cubic shape, the pressure cell may be of any geometric shape suitable for providing the windows and pathways as described herein and amenable to sealing at high pressure. While exemplary fluids are described herein, the invention is not limited to use of any particular pressure and/or temperature transmitting fluid, although preferably the fluid has a relatively low viscosity, is not corrosive to any of the materials in contact therewith, is optically clear, and is weakly scattering with respect to the wavelength(s) of light relevant to its intended use. While exemplary systems are described, including interfaces between the fluid and the sample enclosure, and the fluid and the means for creating the desired temperature and pressure, the invention is not limited to any particular mechanisms or interfaces, so long as they provide a degree of control over pressure and temperature as desired for the intended use. Finally, while described herein in an embodiment using a cuvette with a stopper, it should be understood that any type of sample enclosure may be provided, so long as it permits pressure and temperature transmission with no mixing between the pressure- and temperature-transmitting fluid(s) and the sample enclosed therein.

In an exemplary embodiment, the one or more characteristics of the scattered light is indicative of a shape, a size, degree of aggregation, a plurality of interactions among one or more molecules of the biological sample 140, or a combination thereof. In this way, a contribution of the biological sample 140 to the one or more characteristics of the scattered light can be expressed by the excess Rayleigh ratio

$$( R \frac{ex}{90 °} ) ,$$

as expressed below:

$$R \frac{ex}{90 °} = {C}_{C \& P} \frac{( {V}_{samp} - {V}_{buffer} )}{( {V}_{laser} - {V}_{dark} )} \qquad Equation 1$$

wherein C_{C&P} is a configuration and pressure-specific constant related to geometry of the scattered light, a material of the plurality of windows 136, a pressure, and a toluene calibration standard; Vₛₐₘₚ and V_{buffer} comprise a voltage of the biological sample 140 and a buffer of the biological sample 140, respectively; and Vₗₐₛₑᵣ and V_{dark} comprise a voltage of an incident laser beam 116 and its dark offset, respectively.

The one or more characteristics of the scattered light is affected or influenced by at least the predetermined condition provided by the external condition-inducing system 160/170. In an exemplary embodiment, when the biological sample 140 comprises a solution having monoclonal antibodies, the predetermined condition includes an extended incubation profile. The extended incubation profile comprises subjecting the biological sample 140 to a pattern of being pressurized and depressurized for a ramp rate of up to 100 MPa/minute, a hold period at each 100 MPa interval, and then incubated for up to three days at 300 MPa. In another example, the predetermined condition includes a pressure cycling profile. The pressure cycling profile comprises subjecting the biological sample 140 to a pressure cycle of being pressurized at a ramp rate of up to 100 MPa/minute and up to 300 MPa, incubated for 30 minutes, depressurized up to 100 MPa/minute and down to 0.1 MPa, and incubated for another 30 minutes. Additionally, or optionally, the pressure cycling profile comprising repeating the pressure cycle ten times over an 11-hour period. In still another example, the pressure cycling profile comprises subjecting the biological sample 140 to a pressure cycle of being pressurized at a ramp rate of up to 10 MPa/second and held at 3 minutes, and wherein the predetermined condition includes the temperature set at 20° C. Additionally, or optionally, the pressure cycling profile comprises repeating the pressure cycle 100 times over an 11-hour period. Still further, in an example, the predetermined condition includes a pressure profile. The pressure profile includes subjecting the biological sample 140 to a pattern of being pressurized and depressurized at a ramp rate of 20 MPa/minute until a predetermined pressure is reached, and incubated at 300 MPa for up to three weeks.

In an exemplary embodiment, when the biological sample 140 comprises a solution a low concentration lysozyme sample (c₂), the excess Raleigh ratio

$$R \frac{ex}{90 °}$$

can be regressed as a function of concentration according to:

$$\frac{R \frac{ex}{90 °}}{K ( P )} = {M}_{w , app} {c}_{2}^{2} + {M}_{2} {G}_{22} {c}_{2}^{2} \qquad Equation 2$$

in order to obtain a Kirkwood-Buff integral (G₂₂) value of net protein-protein interactions (PPIs), wherein M_{w,app} and M₂ are the apparent and true molar masses of the protein, respectively, and wherein optical constant K is defined as:

$$K ( P ) = \frac{4 π {n}_{o}^{2}}{{N}_{A} {λ}^{4}} {( \frac{dn}{dc} )}^{2} \qquad Equation 3$$

wherein Nₐ represents Avogadro's number, λ represent a wavelength of the laser source, and nₒ is a solvent refractive index. In this embodiment, a relationship between a second virial coefficient (B₂₂) and G₂₂ can be expressed as G₂₂=−2B₂₂. Additional details of this embodiment is discussed in the Example below.

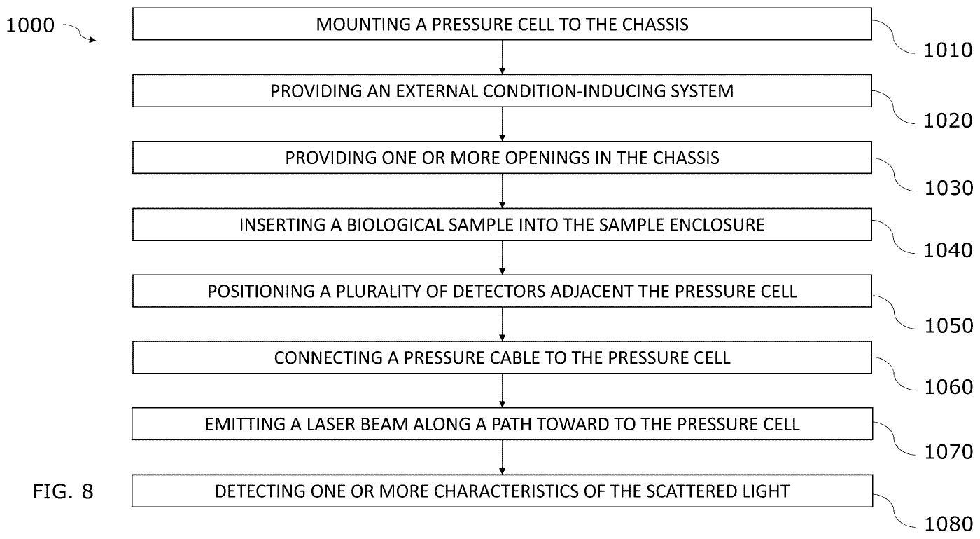

Finally, a method of method of for retrofitting an atmospheric light scattering instrument is disclosed in FIG. 8. The atmospheric-pressure light scattering instrument 110 has a chassis 112 and a laser source 114 configured to emit a laser beam along a path. Additional details of the method 1000 is discussed in view of the components of system 100 and apparatus 120, as discussed throughout the specification. The apparatus 120 is configured to be secured to a light scattering instrument 110 having a chassis 112 and a laser source 114. The method 1000 generally include a step 1010 of mounting a pressure cell to the chassis; a step 1020 of providing an external condition-inducing system; a step 1030 of providing one or more openings in the chassis; a step 1040 of inserting a biological sample into the pressure cell; a step 1050 of positioning a plurality of detectors adjacent the pressure cell; a step 1060 of connecting a pressure cable or tube of the external condition-inducing system to the pressure cell; a step 1070 of emitting a laser beam toward to the pressure cell; and a step 1080 of detecting one or more characteristics of a scattered light.

In step 1010, a pressure cell is mounted to the chassis. In one example, the pressure cell 130 has a chamber defined by one or more walls and configured to contain a fluid pressurized up to 350 MPa. The pressure cell 1330 also includes a sample enclosure 132 comprising a cuvette 134.

The pressure cell 130 also has a plurality of light-transmissive windows 136 in the one or more walls, including at least a beam entry window and a beam exit window positioned in the path of the laser beam 116, and one or more windows positioned orthogonal to the path of the laser beam 166. Additionally, or optionally, a sample enclosure 132 is positioned in the path of the laser beam 116, such that the sample enclosure 132 is isolated from, but in temperature and pressure communication with the fluid in the chamber defined by the pressure cell 130.

In step 1020, an external condition-inducing system is provided. In an example, the external condition-inducing system 160/170 is configured to provide a predetermined condition. Additionally, or optionally, the predetermined condition includes a predetermined temperature and pressure of the fluid within the chamber of the pressure cell 130.

In step 1030, one or more openings in the chassis is provided. In one example, the one or more openings are provided in the chassis 112, and the plurality of holes is configured for facilitating a connection between the pressure cell and the external condition-inducing system of the pressure cell 130 and the external condition-inducing system 160/170. Additionally, or optionally, the one or more openings is insulated and sealed.

In step 1040, the biological sample is inserted into the pressure cell. In an example, the biological sample 140 is inserted into the sample enclosure 132 of the pressure cell 130.

In step 1050, a plurality of detectors is positioned adjacent the pressure cell. In one example, the plurality of detectors 150 are positioned adjacent the pressure cell. The plurality of detectors 150 has one or more of a transmission detector 152 disposed adjacent the beam exit window, and one or more light scattering detectors 154 positioned adjacent the one or more windows 136 orthogonal to the path of the laser beam 116 and configured to detect one or more characteristics of light scattered from the laser beam 116 passing through the sample 140. Additionally, or optionally, the one or more light scattering detectors 154 include a dynamic light scattering detector (DLS) 154b, a static light scattering detector (SLS) 154a, or a combination thereof. 130 and along an orthogonal orientation relative to the transmission detector 152.

In step 1060, a pressure cable of the external condition-inducing system is connected to the pressure cell. In an example, the pressure cable 166 of the external condition-inducing system 160/170 is connected to the pressure cell 130 via a pressure inlet of the pressure cell 130.

In step 1070, a laser beam is emitted along a path toward to the pressure cell. In one example, the laser beam 116 enters the sample enclosure 132 of the pressure cell 130, such that the light scattered by the biological sample 140 is transmitted through the plurality of windows 136.

In step 1080, one or more characteristics of said scattered light is detected by the plurality of detectors 150.

In an exemplary embodiment, method 1000 includes calibrating the apparatus 120. In this arrangement, the biological sample 140 comprises toluene and the adapter is movable along an axis of the chassis 112 for alignment relative to the laser source 114. The toluene is inserted into the pressure cell 130 and is used as a function of high pressure, related to the solid angle subtended by the 90° detector, the pressure chamber material of construction, and cuvette alignment, as expressed by the following equation:

$${R}_{90 °} = {C}_{SSCC} \frac{( {V}_{90 °} - {V}_{90 ° , dark} )}{( {V}_{laser} - {V}_{laser , dark} )}$$

where C_{SSCC} is a Solvent Specific Calibration Constant, V90° and V90° dark are the 90° detector signal voltage and its dark offset voltage, respectively, and Vlaser and Vlaser,dark are the laser monitor signal and its dark offset, respectively. Pure filtered toluene has a Rayleigh ratio (R_{90°}) of 1.406×10−5 cm−1 at a wavelength of 632.8 nm.

### EXAMPLE

The co-inventors assessed feasibility and functionality of the components of the devices, methods, and systems as disclosed herein, as well as verified any updates or improvements made. The prototype devices, methods, and systems were subjected to various tests as detailed herein.

### Solution Preparation

The buffer solution used to prepare the MAb samples was made by dissolving 10 mM L-histidine monohydrochloride monohydrate (Sigma, St, Louis, MO) in distilled, deionized water (resistivity 18.2 MQ·cm, Elga, Woodridge, IL), with and without added 150 mM sodium chloride (where the molarity M=mol/L). The solution was titrated with 5 M sodium hydroxide (Fisher Scientific, Fair Lawn, NJ) to pH 6.5±0.05. Buffer solutions were filtered prior to use (0.22 μm Polyvinylidene Fluoride filters, Chemglass Life Sciences). 50 mM acetate buffer was also prepared for use with lysozyme at pH 4.6 with and without added 150 mM sodium chloride using this method. Histidine was used as a buffer due to its minimal pH shift at elevated pressures and sub-zero° C. temperatures. Zwitterionic buffers such as histidine tend to maintain their buffering ability relatively well at higher pressures, potentially limiting the impact of changes in ionic strength.3 Acetate was chosen for lysozyme solutions to maintain consistency with previous literature.

MAb1 stock solution was received from Amgen Inc. (Thousand Oaks, CA) at a concentration of 30 mg/mL, while MAb2 was supplied by W.L. Gore & Associates, Inc. (Newark, DE) at a concentration of 15 mg/mL. MAb1 and MAb2 are both IgG1 immunoglobulins; each were received in the form of a monomeric solution (greater than 98% monomer measured using size-exclusion chromatography) and used without further purification. MAb stock solutions were dialyzed using 10 kDa molecular weight cutoff (MWCO) dialysis membranes (Spectrum Laboratories, Rancho Dominguez, CA) against the 10 mM L-histidine-HCl buffer. The solution was subsequently filtered with 0.22 μm low-protein-binding filters (Millipore, Billerica, MA). High purity hen egg white lysozyme (recrystallized) in powder form was obtained from Fisher BioReagents (Pittsburgh, PA). A low concentration stock solution (˜15 mg/mL) was prepared by dissolving the lysozyme dried powder into previously prepared pH 4.6 buffer. Samples were gently stirred at room temperature for two hours and then filtered. Protein concentration was determined using UV spectrophotometry (Agilent Technologies-8453 instrument, Santa Clara, CA) absorbance at 280 nm, with extinction coefficients of 1.586 mL mg⁻¹ cm⁻¹, 1.39 mL mg⁻¹ cm⁻¹, and 2.64 mL mg⁻¹ cm⁻¹ for MAb1, MAb2, and lysozyme60, respectively. Gravimetric dilution was used to obtain matching protein concentrations for each MAb and lysozyme, ranging from 1 mg/ml to 10 mg/mL. All samples were degassed prior to HPLS measurements.

### Inventive High-Pressure Light Scattering Apparatus (HPLS)

The HPLS uses an HP cell that allows for quick sample change, reliable separation between the sample and pressure transmitting fluid, and optically compatible windows and cuvette.

### Pressure Cell

The pressure cell, such as ISS model HP-200, available from ISS Medical DBA ISS Inc. of Champaign, IL, includes four windows, each made of quartz, and located at 90° angles relative to each other. With quartz windows, the cell design has a maximum operating pressure of 300 Mega-Pascal (MPa). The pressure cell was mounted on an aluminum adapter 122 configured to fit inside the sample compartment of an atmospheric-pressure light scattering apparatus. The main pressure inlet is located on the top of the cell and was connected to a manual pressure pump, such as one available from High Pressure Equipment Co. of Erie, PA. Pressure was measured with a digital pressure gauge, such as one available from Cooper Instruments & Systems of Warrenton, VA, which is positioned between the pump and the cell. A polyethylene glycol-water mixture, such as one available from PolyScience of Niles, IL, was pumped in a closed circuit through internal flow channels of the cell, and demonstrated to achieve temperatures between −20° C. and 90° C. A square bottom quartz cuvette (volume requirement of ˜0.3 mL) was used for the sample enclosure. A Teflon stopper was prepared to fit the cylindrical bottleneck of the cuvette and allowed enough travel distance on the neck to compress downward when pressure is applied. Distilled, deionized water (resistivity 18.2 MQ·cm, Elga) was filtered and degassed to be used as the pressurizing medium.

### Light Scattering Apparatus

The pressure cell was bolted into an atmospheric-pressure light scattering instrument equipped with a 658 nm, 50 mW laser, such as one available from Wavespectrum, Inc of Beijing, China. A silicon PIN photodiode, such as model 10698-3, as available from UDT Sensors Inc., of Hawthorne, California, was used as a static scattering detector and was chosen for low noise and fast response. The resulting analog signal was converted with a 24-bit analog-to-digital converter. Eight holes were drilled into the adapter 122, four to be mounted onto the bottom of the pressure cell, and the other four to be mounted onto the steel sub-chassis inside the instrument to allow for vertical motion during alignment. Once vertically aligned, this arrangement allowed for laser entry (0° to incident), beam scatter detection (90°), and transmission detection (180°). Holes were drilled in the side of the light scattering instrument for tubing egress to accommodate heating/cooling liquid. The holes were insulated and sealed to any light. Another hole was drilled through the top of the unit to allow the pressure cable to pass through to the top plug of the pressure cell. Briefly, 10 nm gold nanoparticle samples (NIST RM8011, Gaithersburg, MD) were analyzed to assess system performance. Triplicate Au nanoparticle samples were incubated at both atmospheric pressure (0.1 MPa) and high pressure (300 MPa) to evaluate detector drift (less than 1%) and sample repeatability (within 1% variation, cf. Supporting Information). Additionally, toluene was used to calibrate the given instrumentation arrangement as a function of high pressure, related to the solid angle subtended by the 90° detector, the pressure chamber material of construction, and cuvette alignment.

### High Pressure Sample Incubation

Batch isothermal and isobaric SLS measurements were carried out using the HPLS instrument. Monomeric samples were filtered and centrifuged before measurements to reduce dust and particulate artifacts. Matching buffer was measured for each sample and solvent background scattering was subtracted from the protein solution signal (Equation 1). To ensure the light scattering signal consisted only of contributions from the protein, additional background sources including stray light and dark offset were subtracted from the total intensity. After the subtraction, the signal was normalized for variations in laser pathlength, detector drift, and solution density as a function of pressure. Pressurizing the cell causes an outward force on the exterior windows. Although the windows are set in place between stainless steel surfaces, the O-ring seals slightly compress under pressure thereby changing the light pathlength across the reservoir holding the pressure transmitting fluid. This effect was small but non-trivial. The contribution of the protein to total scattered light was expressed by the excess Rayleigh ratio,

$$R \frac{ex}{90 °} ,$$

which is expressed as follows:

$$R \frac{ex}{90 °} = {C}_{C \& P} \frac{( {V}_{samp} - {V}_{buffer} )}{( {V}_{laser} - {V}_{dark} )} \qquad Equation l$$

where C_{C&P} is the configuration and pressure-specific constant related to scattering geometry, window material, pressure, and toluene calibration standard. Both Vₛₐₘₚ and V_{buffer} refer to the voltage on the detector measured with the sample and buffer, respectively. Additionally, both Vₗₐₛₑᵣ and V_{dark} are the detector voltages of the incident laser and its dark offset, respectively.

For the extended incubation studies, MAb1 and MAb2 were pressurized and depressurized no faster than 100 MPa/minute, with hold steps at each 100 MPa interval, then incubated for up to three days at 300 MPa, during which the static light scattering signal was measured. HPLS data were measured immediately upon reaching a pressure set-point, as well as after the solution was allowed to equilibrate for 30 minutes.

Pressure cycling experiments were conducted using similar ramp and hold periods. Similar to the pressurization rate used for HPLS incubations, samples were pressurized no faster than 100 MPa/minute up to 300 MPa (with no equilibration period at lower pressure intervals) and incubated for 30 minutes. The sample was depressurized at a similar rate until ambient pressure was reached (0.1 MPa), at which point samples were incubated for another 30 minutes completing one cycle. Cycles were repeated ten times over an 11-hour period. HPLS measurements were binned (e.g., time averaged every five minutes) to average minor oscillations in light scattering profiles over the time period.

A Barocycler 2320 EXT, such as one available from Pressure BioSciences Inc. of Medford, MA, was then used to conduct 100 pressure cycles over the same 11-hour time period (ramp at no faster than 10 MPa/second, hold at 3 minutes), followed by ex situ aggregation characterization with the HPLS. The Barocycler sample chamber temperature was controlled at 20° C. with an external water bath. Samples were injected into a microtube and then capped with a microcap. Microtube and microcap materials of construction were inert and retain integrity while being malleable to transmit pressure in the relevant ranges. Overall, samples were pressure cycled 100 times in 10% of the time period (1.1 hours) using a pressure ramp of 100 MPa/second and holding for 30 seconds.

Ex situ Aggregation Quantitation

The Barocycler 2320 EXT was also used to conduct HP (300 MPa) incubations on triplicate samples for MAb1 and MAb2. Samples were (de) pressurized at 20 MPa/minute until the set point was reached and then incubated at 300 MPa for up to three weeks. Size exclusion chromatography (SEC) was used to quantify monomer fraction ex situ (i.e., after the pressure incubation). Prior to injection onto the column, samples were quenched by returning to ambient pressure/temperature conditions in order to arrest the aggregation process. This was confirmed by measuring the same sample by SEC at multiple timepoints beyond the end of incubation (i.e., after the sample was returned and held at ambient conditions). An Agilent 1100 series instrument (Agilent Technologies, Wilmington, DE) consisted of a variable wavelength detector (VWD) with protein absorbance measured at 280 nm, connected in-line to a Tosoh (Montgomeryville, PA) TSK-GEL G3000SWxl column. Injection volumes contained approximately 30 μg of protein. The mobile phase had a flow rate of 0.75 mL/min and consisted of 0.5% volume fraction of phosphoric acid (Fisher Scientific) at pH 5.0 and 100 mM NaCl in distilled, deionized water. Samples were centrifuged at ˜10,000 relative centrifugal force for five minutes prior to injection. The monomer fraction was calculated by the ratio of the area of the sample absorption peak and the area of a control absorption peak measured at an initial time point.

### Second Virial Coefficient Measurements at High Pressure for Lysozyme

For low concentration lysozyme samples (c₂) that showed no aggregation (and therefore not applicable for MAb solutions), the excess Raleigh ratio,

$$R \frac{ex}{90 °}$$

was regressed as a function of concentration using Equation 2 to obtain the Kirkwood-Buff integral (G22) values of net protein-protein interactions.

$$\frac{R \frac{ex}{90 °}}{K ( P )} = {M}_{w , app} {c}_{2} + {M}_{2} {G}_{22} {c}_{2}^{2} \qquad Equation 2$$

In Equation 2, M_{w,app} and M₂ are the apparent and true molar masses of the protein, respectively. The optical constant K is defined by Equation 3 below.

$$K ( P ) = \frac{4 π {n}_{o}^{2}}{{N}_{A} {λ}^{4}} {( \frac{dn}{dc} )}^{2} \qquad Equation 3$$

where Nₐ represents Avogadro's number, λ the laser wavelength, and nₒ the solvent refractive index. The optical constant is subject to change with pressure, since the differential refractive index increment, dn/dc has a pressure dependence. At sufficiently low c2 and in weak protein-protein interactions (PPI) conditions, B₂₂ is related to G₂₂ via the relationship G₂₂=−2B₂₂. B₂₂ is a quantitative measure of deviations from ideality resulting from PPI, where a positive (negative) value corresponds to net repulsive (attractive) PPIs. A B₂₂ value for lysozyme obtained at atmospheric pressure using a Wyatt Dynapro Nanostar (Wyatt Technology, Santa Barbara, CA) was in close agreement with the value obtained at room pressure using the inventive HPLS apparatus or system.

### Results and Discussion

### High Pressure Aggregation and Qualitative Assessment of PPIs

The excess Raleigh scattering ratio

$$R \frac{ex}{90 °} / K$$

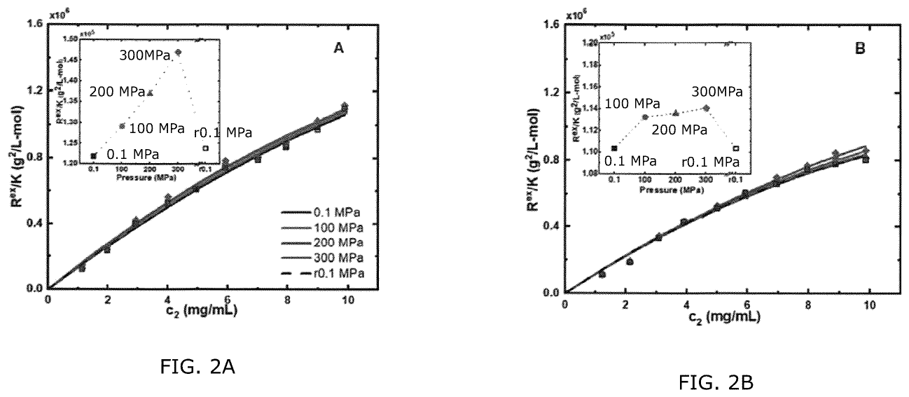

was calculated and plotted versus protein concentration for MAb1 and MAb2 at various pressures (FIGS. 2A-2B). FIGS. 2A-2B illustrate excess Rayleigh scattering profiles for a concentration series (1-10 mg/mL) incubated up to 300 MPa in 100 MPa increments of MAb1 (FIG. 2A) and MAb2 (FIG. 2B) prepared in IOmM histidine buffer at pH 6.5 (lowest ionic strength, as no salt added). Isobaric lines connect concentrations and are based on the fit with Equation 2. Samples were measured at atmospheric pressure of 0.1 MPa (squares), 100 MPa (circles), 200 MPa (triangles), and 300 MPa (diamond), and return to atmospheric pressure (open black squares connected by a dotted line). The insets show

$$R \frac{ex}{90 °} / K$$

of the 1.2 mg/mL sample incubation as a function of pressure for comparison. The dotted lines in the inset are used to guide the eye to follow the pressurization/depressurization cycles applied to the sample.

Previous work characterized conformational stability of these MAbs at high pressure revealing partial unfolding for both proteins, particularly for MAb1, for a range of pressure/temperature conditions. Symbols correspond to the experimental data and the lines are fits to Equation 2 using G₂₂=−2B₂₂. The calculated B₂₂ values for Mab1 and MAb2 at ambient pressure were (10.6±0.6) ml/g and (11.9±1.7) mL/g, respectively. The observed downward curvature of the Rayleigh scattering profile at atmospheric pressure and corresponding positive B₂₂ value indicate net repulsive PPIs. This downward curvature is similar in magnitude for the profiles at different pressures, suggesting net PPI are equivalent in at least magnitude and sign across pressures (and MAbs). Under conditions in which aggregation was observed (indicated by an increase in scattered light intensity) B₂₂ values are not reported. The insets in both panels of FIGS. 2A-2B demonstrate the increase in excess Rayleigh intensity as a function of pressure for the 1.2 mg/ml sample. For both proteins, as concentration increased, the difference in observed intensity from the sample at ambient pressure of 0.1 MPa to high pressure at 300 MPa became slightly larger. For polydisperse, ideal solutions of small protein size, the Rayleigh ratio is proportional to the sum of Rayleigh ratios of proteins at each possible molar mass (and corresponding oligomer mass). The individual Rayleigh ratios are the product of the square of the molar mass of the ith species and the number concentration of that species. It follows that larger particles give stronger scattering despite the decrease in number concentration of available scattering particles. However, in practice, deconvoluting the light scattering signal into monomer and aggregate species components is complicated by the formation of large-sized aggregates.

In cases where aggregation was irreversible for MAb1, the presence of the dimer form in solution was confirmed when post-incubated samples were measured with SEC. MAb2 aggregation at high pressure was reversible at the time scales analyzed (sub-hour) and was not detected with SEC. B₂₂ is formally related to protein-protein interactions in the low c2 limit when integrating over all spatial degrees of freedom of any co-solute or cosolvent species and solvent via the potential mean force W₂₂, between precisely two monomer species. The integration is only valid if aggregates are not present, a condition not met at elevated pressure for these MAbs. Therefore, the fits at higher pressures will exclude multibody interactions (i.e., aggregate species), convoluting the quantitation of B₂₂. The downward curvature of the fits at high pressure is nonetheless apparent, likely indicating net repulsive interactions, in lieu of the small amount of dimerization and oligomerization. Mechanistically, this results in more oligomerization events for MAb1 at comparatively higher pressure, likely favored by a larger net partial specific molar volume decrease in the resulting species. Pressures up to 300 MPa are not enough to overcome net repulsive forces that remain despite partial unfolding of the monomer for these MAbs.

### Extended Incubation at High Pressure

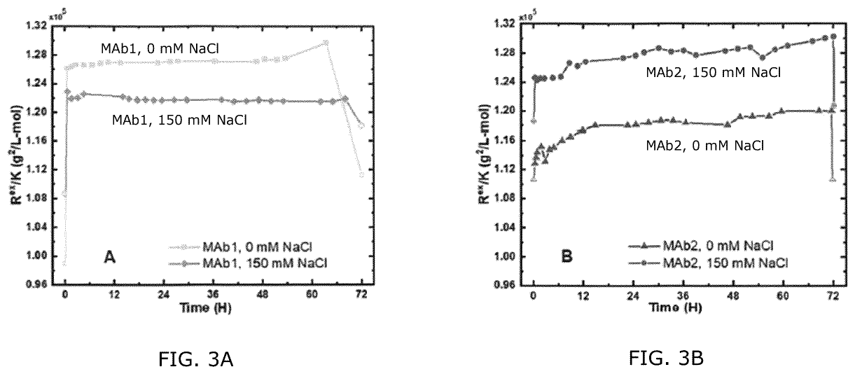

Samples were incubated at 300 MPa, 20° C. for up to three days in the inventive HP light scattering apparatus. In situ measurements of scattering intensity were taken in intervals of 2-3 hours for both MAbs at low and high total ionic strength (FIGS. 3A-3B). FIGS. 3A-3B illustrate excess Rayleigh scattering ratio as a function of time starting at atmospheric pressure (open symbols) and incubated at 300 MPa (closed symbols) for 72 hours. FIG. 3A shows MAb1 and FIG. 3B shows MAb2, each at 1 mg/mL with 0 and 150 mM NaCl were held at 20° C. for the duration of the incubation. A discernably different extent-of-aggregation was observed with these MAbs. The extent of aggregation can be gauged by the increase in the Rayleigh ratio. For MAb1 solutions at 1 mg/mL for the low ionic strength condition, the initial increase in signal was (27.3±1.0) % larger than that of ambient pressure, while the high ionic strength condition produced a (13.0±0.2) % signal increase. These results for the initial time point agreed with measurements shown in FIG. 2A.

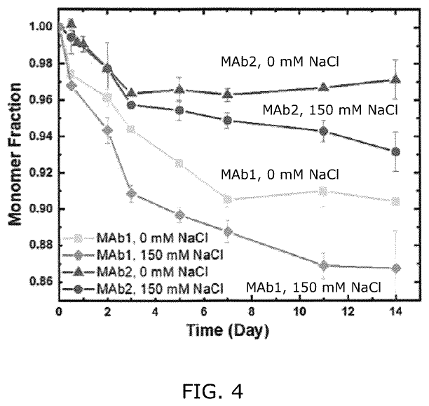

Over time, the signal for MAb1 tended to increase for the low ionic strength condition while it remained steady in the high ionic strength solution. Both MAb1 samples gave substantially higher signals when returned to ambient pressure, indicating the presence of irreversible aggregates, confirmed with monomer loss profiles measured using SEC (FIG. 4). The initial signal increase upon pressurization for MAb2 was (2.0±0.1) % and (5.1±0.1) % for 0 mM and 150 mM NaCl solutions, respectively. This initial signal increase matched results of the signal increase observed from the concentration series as shown in the inset of FIG. 2B. In both solution conditions for MAb2, there was a steady increase in the excess Rayleigh ratio over the 72-hour period. When the samples were depressurized, the signal returned nearly to the starting value. Compared to MAb1, MAb2 showed less monomer loss as a result of pressure incubations (FIG. 4).

FIG. 4 illustrates monomer loss profiles from high pressure incubations for 1 mg/mL MAbs at pH 6.5 (10 mM histidine buffer). Samples were held at 300 MPa, 20° C. for up to 14 days. Error bars correspond to the standard deviation of triplicate samples. In particular, the results shown in FIG. 4 reveal that aggregation was substantially accelerated at high pressure, compared to corresponding atmospheric pressure control samples which showed minimal aggregation. The aggregation rates depicted in FIG. 4 increased for solutions with elevated total ionic strength. The extent of aggregation and initial rate were higher for MAb1 compared to MAb2 (all sample conditions). As the protein aggregated into dimers in the case of MAb1 and insoluble aggregates in the case of MAb2 (confirmed with SEC chromatograms) monomer fraction in solution decreased, reaching effective plateaus of nearly 0.86±0.03 and 0.91±0.01 for MAb1 high and low ionic strength samples, respectively. Several possible mechanisms of baro-sensitivity (the process of (de) stabilizing monomer at high pressure) have been considered to explain the difference in aggregation behavior observed between low and high total ionic strength conditions. The effective charge on the surface of a protein can change with pressure due to electrostriction effects and through pKa shifts of amino acid side-chains and preferential hydration of salt-ions. An increase of the absolute surface net charge increases electrostatic PPIs.

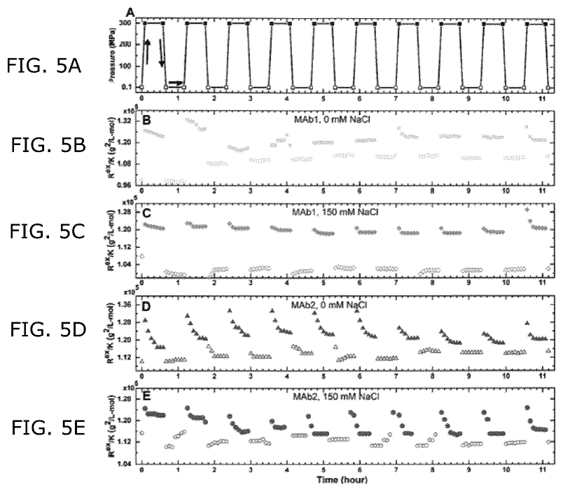

As a result, net interprotein interactions (either repulsive or attractive) can become stronger at high pressure. Although this is not expected to be the case for one pressurization cycle (cf. FIG. 2), it may explain aggregation behavior after multiple pressure cycles where there may be a kinetic effects since each cycle and hold was for a finite time (cf. FIG. 5). The monomer loss of pressure incubated solutions of MAb1 with added NaCl is greater than monomer loss for solutions without NaCl. The opposite trend was observed based on in-situ light scattering, in which MAb1 solutions without NaCl gave larger increases in the excess Rayleigh scattering ratio. However, the extent of in situ reversible aggregation varied depending on the solution total ionic strength. Higher concentrations of NaCl are known to cause electrostatic screening effects, yet these can be disrupted at high pressure due to the electrostriction effect. If that were the case here, weakening the screening of repulsive interactions would result in less aggregation, which was not observed. Alteration in the hydration state of the protein may result in changes in particle size unrelated to structural changes, but dependent on total ionic strength.

### Pressure Cycling Effects on Aggregation and Stability

MAb1 and MAb2 aggregation caused by pressure cycling was analyzed with HPLS and SEC to investigate the effects of pressurization rate, hold time, and number of cycles on MAb aggregation behavior. FIGS. 5A-5E shows the excess Rayleigh ratio determined for MAb1 and MAb2 during pressure cycling. FIG. 5A depicts pressure cycle parameters, e.g. samples were pressurized at 1 MPa/second and held at high pressure for 30 minutes (closed symbols). Next, the samples were depressurized at 1 MPa/second and held for 30 minutes at ambient pressure of 0.1 MPa (open symbols) completing one cycle. FIGS. 5B-5E illustrate excess Rayleigh scattering shifts induced by 10 pressure cycles up to 300 MPa at 20° C. on MAb1 and MAb2 samples prepared at 1 mg/mL, 10 mM histidine pH 6.5 with and without an addition of NaCl (150 mM). The value of pressure at a given time in the cycle is detailed in FIG. 5A.

The pressure is aligned with the other panels to clearly demonstrate its given value at a particular time and light scattering measurement. For MAb1 in a low ionic strength solution (FIG. 5B), there was a large initial jump in scattering intensity observed immediately when 300 MPa was achieved. This intensity peak

$$( R \frac{ex}{90 °} / K )$$

began to decrease while pressure was held at 300 MPa and then it leveled at 1.23 g²/L-mol, consistent with a certain extent of reversibility of the HP effects. As the pressure cycling continued, the gap between scattering intensity at low and high pressure narrowed. Scattering intensity at ambient pressure increased toward the scattering intensity measured at 300 MPa (which was slightly decreasing) as the cycle count increased. This suggested pressure induced creation of reversible, partially-unfolded intermediate states that gradually aggregated into dimer with each repeated cycle. The aggregation process was still limited; once the scattering baseline plateaued at atmospheric pressure, partially unfolded monomer did not continue to irreversibly aggregate at high pressure. With most monomer loss occurring during the first two pressure cycles, the initial cycles may remove the most aggregation-prone charge variant(s).

Here, ex situ quantification of monomer loss also confirmed this result at increased cycling counts, which showed extent of aggregation to be wholly dependent on the cycle count itself, and not the pressurization rate or hold times (up to 30 minutes), which were varied over several orders of magnitude. FIG. 5C shows pressure cycled light scattering intensity for MAb1 in a high total ionic strength solution. Interestingly, the high total ionic strength solution exhibited a similar behavior to its low ionic strength counterpart in terms of the high- and low-pressure baselines converging, but the intensity gap for the sample containing 150 millimolar (mM) NaCl remains substantially larger after ten pressure cycles. This result suggests that there is a higher concentration of irreversible dimer aggregate species at high pressure (confirmed with ex situ SEC). Compared to the monomer loss during high pressure incubation of MAb2 (c.f. FIG. 4), MAb1 produced a low quantity of dimer at high pressure conditions that began to form larger irreversible oligomers and other insoluble aggregates at a fast initial rate.

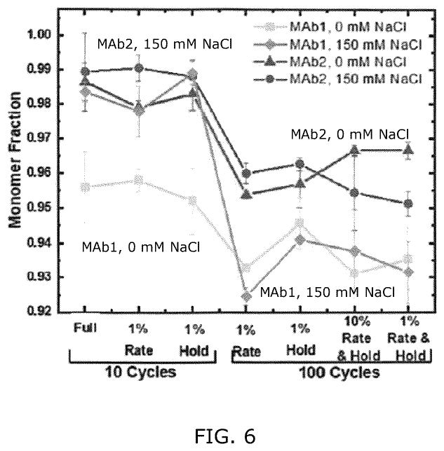

MAb2 solutions with and without additional 150 mM NaCl presented similar behavior when measured with the inventive HPLS, yet were markedly different from MAb1, particularly in extent of reversibility and baseline (ambient pressure) consistency. Specifically, FIGS. 5D and 5E show that upon pressurization, the light scattering signal sharply spikes indicating a transient presence of higher molecular weight species (not necessarily dimer, as this was not confirmed with SEC). This signal increase is short lived, and the signal levels off closer to the low-pressure baseline. The light scattering signal at ambient pressure still tended to increase, albeit slightly, with each cycle indicating the increased presence of aggregate that was irreversibly formed at high pressure. Even at ambient pressure, a variable and increased signal was observed towards the beginning of the 30-minute hold, though the signal plateaued after several minutes. As shown in FIG. 6, a smaller degree of monomer loss was observed for MAb2 compared to that of MAb1 after ten pressure cycles. For all samples, there was a uniform decrease in monomer fraction with an increase in cycle count. When the cycle rate was accelerated, monomer fraction remained relatively consistent for all samples, suggesting the number of high/low pressure cycles—not pressurization rate—was the key factor. In particular, FIG. 6 shows monomer loss profiles from pressure cycle incubations (0.1 to 300 MPa) of MAb1 and MAb2 samples prepared at 1 mg/mL 10 mM histidine pH 6.5 at low and high total ionic strength. Error bars correspond to the standard deviation of triplicate samples. Three incubations were 10 pressure cycles at a ‘Full’ pressurization rate of 20 MPa/minute and hold time of 30 minutes. Other samples are labeled based variations of the ‘Full’ pressurization rate and hold times.

### Quantifying PPIs of Lysozyme at High Pressure

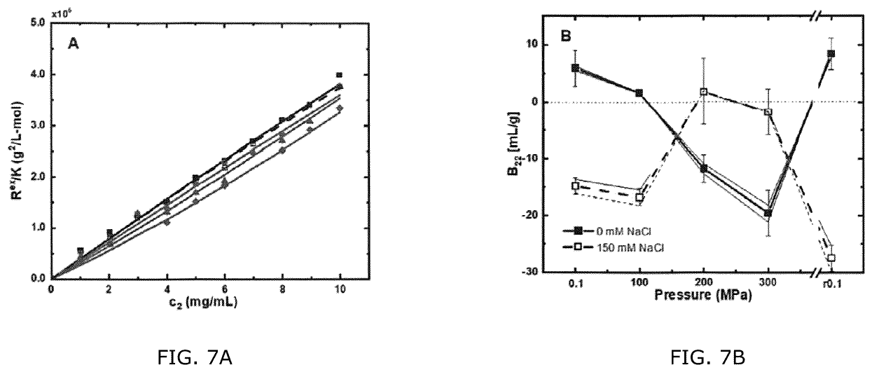

FIGS. 7A-7B illustrate excess Rayleigh scattering profiles for a concentration series of lysozyme prepared in 50 mM acetate buffer, at pH 4.6 (1-10 mg/mL) incubated up to 300 MPa in 100 MPa increments, with isobaric lines corresponding to the fit with Equation 3. Samples were measured at ambient pressure of 0.1 MPa (squares), 100 MPa (circles), 200 MPa (triangles), 300 MPa (diamond), and a return to 0.1 MPa (open squares). The osmotic second virial coefficient (B₂₂) as a function of pressure with O (solid square) and 150 mM NaCl (open square) samples, and with lines to guide the eye. Error bars correspond to the 95% confidence interval of the fit parameter. The range around the points shows the minimum and maximum B₂₂ calculated for estimated extreme dn/dc values.

Static light scattering was also used to determine

$$R \frac{ex}{90 °} / K$$

as a function of protein concentration and pressure for lysozyme.

$$R \frac{ex}{90 °} / K$$

is plotted as a function of c₂ for low concentrations (between I and 10 mg/mL) in isobars up to 300 MPa (FIG. 7A). Symbols corresponding to experimental data were fit to Equation 2 in a similar procedure described for MAb1 and MAb2. Unlike the MAbs, lysozyme did not aggregate at high pressure, a result that is consistent with HP data found in literature. For lysozyme, a slight decrease in the intensity of light scattered at elevated pressure was observed for each concentration. This disparity grew larger at higher concentrations, and was nearly indistinguishable from 1 to 3 mg/mL. The

$$R \frac{ex}{90 °} / K$$

profiles at each pressure are distinct, which suggests a shift in net PPIs as a function of pressure. More specifically at low pressure, a slightly downward curvature is observed, corresponding to weak net repulsive PPIs. Upward curvature observed in the

$$R \frac{ex}{90 °} / K$$

profile at 300 MPa corresponds to net attractive PPIs.

The pressure dependence of dn/dc has the potential to impact the quantitation of B₂₂ resulting from the fit in Equation 2. The refractive index is broadly defined as the degree to which the path of light is bent when traversing media boundaries (assuming isotropic media). On an atomistic scale, the refractive index results from the polarizability of atoms due to shifts in local electron configuration. As a consequence, the large-scale structure of the protein has minimal effect on the value of the refractive index. Since pressure has an impact on solvation effects, local polarizability can be perturbed. Amino acids with high polarizability are those that contain aromatic rings or sulfur atoms (particularly amino acids involved in disulfide bonds). Lysozyme is susceptible to shifts in refractive index because under pressure the relative solvent-exposed hydrophobic area increases, shown both experimentally and in silico. Modeling the shift is complicated by the lack of high resolution techniques that estimate solvation effects on exposed hydrophobic residues at high pressure. Deviations in modeled molar refractivity are likely attributed to small shifts in solvation and errors propagated by partial specific volumes of amino acids. Partial specific molar volume is a variable that is closely related to the refractive index increment. The volume change (ΔVᵤₙ) of unfolded lysozyme monomer under pressure is relatively small and can range from −25 to 0 mL/mol depending on the solution conditions (e.g. temperature, pH, etc.). In this study, pressure only partially unfolded lysozyme, and changes in the partial specific molar volume are expected to be minimal in addition to any resulting change in the dn/dc value. The measured dn/dc value of lysozyme at ambient pressure and the buffer conditions used in this study was (0.1922±0.0010) mL/g. When performing the fit in Equation 2, dn/dc values up to three times the modeled shift observed over the maximum potential (ΔVᵤₙ) were input to gauge impact on B₂₂. This conservative range was expected to capture any potential deviation of dn/dc based on modeled shifts as a result of partial unfolding and changes in the partial specific volume induced by temperature or chaotropic reagents. The second virial coefficient was impacted by a maximum of 10% of the initial value at the most extreme dn/dc values. The resulting values of B₂₂ obtained from the fit to Equation 2 for lysozyme at low and high ionic strength are shown in FIG. 7B. The minimum and maximum values of B₂₂ obtained from Equation 2 for a given pressure while testing different extreme values for dn/dc are illustrated as a shaded range around the central point. The central point reflects the base (measured) dn/dc value used, which was corrected in proportion to changes in the refractive index of water as a function of pressure. At the low ionic strength condition, B₂₂ changed sign above 100 MPa and magnitude at pressures up to 300 MPa. A monotonic decrease in B₂₂ was observed as a function of pressure. For the high ionic strength solution, B₂₂ started negative at ambient pressure, indicating net attractive interactions. Electrostatic interactions are screened at these salt concentrations, indicating net PPIs are a combination of nonelectrostatic interactions. B₂₂ also changed sign and magnitude at high pressure, though it trended toward a positive value (FIG. 7B), indicating net attractions weakened with higher pressure. We also considered using a reference point of the B₂₂ value for the steric-only interaction point (B_{22,ST}) as we have advocated previously, but since the main points noted here are focused on the changes in B₂₂ this did not change the next effects or discussion here.

The pressure dependence of the second virial coefficient can follow from shifts in the dissociation constant, pKₐ, of amino acid residues. The magnitude of the charge on the surface of the protein particle will shift as a result, potentially altering the magnitude of interactions. In the present study, acetate buffer (pH 4.6) was selected to maintain consistency with previous research on lysozyme at atmospheric and high pressure. Anionic buffers such as acetate tend to have negative partial specific reaction volumes (ΔV=−11.2 ml/mol) and larger ΔpH/ΔP values (−0.08 pH units/100 MPa). Shifts in the second virial coefficient at elevated pressure can also depend on changes in monomer structure and size induced by either alteration to the protein hydration state or partial unfolding; such conformational perturbations could change both net surface charge and particle size.

Most pressure-induced aggregation and unfolding studies have focused on globular proteins. In this study, lysozyme served as an example to illustrate the possibilities of HP static light scattering in probing reversible protein-protein interactions. Banachowicz previously studied this molecule using a customized HP dynamic light scattering (DLS) instrument. In that work, DLS was used to measure fluctuations in scattering intensity as a function of pressure to determine the translational diffusion coefficient. The diffusion coefficient was regressed vs concentration to produce a first order interaction parameter, k_{D}, for successive pressures. The interaction parameter can often be used as a surrogate for B₂₂, and similarly, a positive (negative) value indicates net repulsive (attractive) intermolecular interactions. However, the change in magnitude and sign of ko in relation to B₂₂ is not exactly matched, particularly in the case of net weak interactions as observed here. To first approximation, the ko measured by Banachowicz scales closely with shifts in B₂₂ described in this study as a function of pressure. Other work has shown different behavior, though there were other excipients present to induce crystallization complicating any comparison in reversible protein-protein interactions.

### CONCLUSION

Static light scattering represents a powerful technique for determining changes in association and dissociation of monomers and PPIs. The former is measured by variations in the intensity of scattered light with time and the latter is observed by calculating B₂₂ (i.e., fitting excess Rayleigh scattering profiles) as a function of pressure at low protein concentration. The inventive high-pressure light scattering apparatus, suitable for protein applications, is similar to typical atmospheric-based light scattering equipment. The current configuration was specified for a large pressure range from ambient up to 350 MPa, and a wide temperature range between −20° C. and 90° C. The inventive HPLS arrangement gives a static light scattering angle of 90° from the incident laser beam (through quartz optical windows). The HPLS design and operating protocols support the advancement of pressure environments applied to protein solutions.

The applicability of the HP cell in performing static light scattering measurements was demonstrated with three proteins: two MAbs and lysozyme. The results show that the inventive HPLS apparatus is sensitive to the corresponding extent of aggregation during in situ pressure incubations, as well as the effects of total ionic strength on solution behavior. The underlying molecular aggregation mechanisms that differentiate the two MAbs are an area for future work. Pressure incubations combined with ex situ analysis showed that resultant monomer loss tended to correlate with the extent of in situ unfolding and aggregation. Combinations of pressure cycling parameters also showed the extent of pressure-induced aggregation. Finally, changes in protein-protein interactions, quantified using the second virial coefficient, B₂₂, as a function of pressure were observed using lysozyme. A monotonic decrease in B22 was observed as a function of pressure for the low ionic strength solution. For the high ionic strength solution, B₂₂ started negative at ambient pressure, indicating net attractive interactions, but trended positive, indicating net attractions weakened with pressure. As a relatively well-characterized protein at high pressure, lysozyme served as a positive-control in validating the instrument performance. The results for lysozyme are consistent with the high-pressure studies available in the literature for this protein. In total, the effects of pressure cycling on MAbs were not all reversible, and not to be interpreted in the context of B₂₂ in a similar fashion as lysozyme. Therefore, static light scattering was shown to be a useful method to investigate protein-protein interactions at elevated pressure because the inventive HPLS allows for the detection of changes in size and shape of proteins (and potential aggregates) with minimal material required for use. This technique and the associated measurements advance the HP toolbox and aid in understanding the link between protein aggregation and PPIs at HP and atmospheric conditions. The technique is useful in studies to assess intermediate states of viruses (or virus-like particles), nucleic acid-based therapeutics, and other multimeric proteins.

Thus, the apparatus, methods, and systems disclosed herein address shortcomings of conventional standard atmospheric pressure and/or temperature setups. These known instruments for evaluating light scattering properties of a liquid sample have limited pressure-temperature ranges, or are not obtainable, adaptable, and/or reproducible with regular means. Further, because extreme temperature and pressure can disturb the carefully constructed and calibrated geometries required by light scattering equipment, investigating the shape, size, and interactions of biomolecules, such as MAbs, in environments with high pressure and/or low temperature, using light scattering techniques difficult. However, the inventors have discovered the disclosed apparatus, methods, and systems discussed herein, which includes a pressure cell capable of achieving pressures up to 350 MPa and temperatures that range from −20° C. to 90° C., allows for study of biomolecules at certain conditions (e.g. high pressure, sub-zero ° C., etc.). The design addressed several challenges created by high pressure environments, including separation between the biological sample and pressure transmitting solution. For example, the inventive pressure cell includes a sample enclosure comprising a low volume cuvette with a pressure-transmitting closure that acts like a piston to transmit pressure while simultaneously separating the internal solution from the pressurizing liquid. In sum, the examples discussed above show the varied applications of the apparatus, methods, and systems disclosed herein.

Although the invention is illustrated and described herein with reference to specific embodiments, the invention is not intended to be limited to the details shown. Rather, various modifications may be made in the details within the scope and range of equivalents of the claims and without departing from the invention.

1. A system for evaluating light scattering properties of a liquid sample, the system comprising:
an light scattering instrument comprising a chassis and a laser source configured to emit a laser beam along a path;
an apparatus configured to be secured to the light scattering instrument, the apparatus comprising:
a pressure cell mounted to the chassis, the pressure cell comprising a chamber defined by one or more walls and configured to contain a fluid pressurized up to 350 MPa, a plurality of light-transmissive windows in the one or more walls, including at least a beam entry window and a beam exit window positioned in the path of the laser beam, and one or more windows positioned orthogonal to the path of the laser beam, and a sample enclosure positioned in the path of the laser beam, the sample enclosure isolated from, but in temperature and pressure communication with, the fluid, the chamber defining a fluid passageway configured to transmit heat or cold from the fluid therein to the biological sample secured within the sample enclosure;
an external condition-inducing system configured to provide a predetermined temperature and pressure of the fluid within the pressure cell chamber;
a biological sample received in the sample enclosure of the pressure cell;
a plurality of detectors positioned adjacent one or more of the plurality of windows, the plurality of detectors comprising one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample.

2. (canceled)

3. (canceled)

4. (canceled)

5. The system of claim 1, wherein the plurality of light-transmissive windows includes two orthogonal windows, and light scattering detectors positioned adjacent said two orthogonal windows.

6. The system of claim 1, wherein the light scattering detector comprises a static light scattering detector (SLS) and a dynamic light scattering detector (DLS) the plurality of light-transmissive windows includes two orthogonal quartz windows, wherein the light scattering detector comprises an SLS and a DLS positioned adjacent orthogonal windows and opposite one another.

7. (canceled)

8. The system of claim 7, wherein the one or more characteristics of the scattered light is indicative of a shape, a size, degree of aggregation, a plurality of interactions among one or more molecules of the biological sample, or a combination thereof, and a contribution of the biological sample to the one or more characteristics of the scattered light can be expressed by the excess Rayleigh ratio
$$( R \frac{ex}{90 °} ) : R \frac{ex}{90 °} = {C}_{C \& P} \frac{( {V}_{samp} - {V}_{buffer} )}{( {V}_{laser} - {V}_{dark} )} ,$$
wherein C_{C&P} is a configuration and pressure-specific constant related to geometry of the scattered light, a material of the plurality of windows, pressure, and a toluene calibration standard; Vₛₐₘₚ and V_{buffer} comprise a voltage of the biological sample and a buffer of the biological sample, respectively; and Vₗₐₛₑᵣ and V_{dark} comprise a voltage of an incident laser beam and its dark offset, respectively.

9. The system of claim 1, wherein the predetermined condition comprises the pressure in a range between ambient pressure to 350 MPa, and the temperature in a range between −20° C. and 90° C.

10. (canceled)

11. (canceled)

12. The system of claim 1, wherein the light-scattering instrument is an atmospheric pressure light scattering instrument and the pressure cell is mounted to the chassis using an adapter translatable along an axis of the chassis for alignment, the alignment permitting entry of a laser beam from the laser source into the pressure cell when mounted on the adapter.

13. (canceled)

14. The system of claim 1, wherein the external condition-inducing system comprises a pump connected to a pressure inlet of the pressure cell, the external condition-inducing system also having a pressure gauge positioned between the pump and the pressure cell, the pressure gauge configured to measure applied pressure to the pressure cell.

15. (canceled)

16. The system of claim 15, wherein the fluid comprises a polyethylene glycol-water mixture capable of reaching a temperature range between −20° C. and 90° C.

17. (canceled)

18. The system of claim 1, wherein the sample enclosure comprises a pressure-transmitting closure disposed between the fluid in the chamber and the sample in the sample enclosure, the pressure-transmitting closure configured to reversibly change in configuration to cause a change in pressure of the fluid to be translated into a proportional change in pressure of the sample.

19. The system of claim 18, wherein the pressure-transmitting closure comprises a stopper configured to translate along a neck of the cuvette to transmit pressure to the biological sample from the external condition-inducing system.

20. (canceled)

21. (canceled)

22. The system of claim 8, wherein the biological sample comprises a solution having proteins.

23. The system of claim 22, wherein the biological sample comprises a solution having monoclonal antibodies.

24. (canceled)

25. The system of claim 23, wherein the predetermined condition includes an extended incubation profile, wherein the extended incubation profile comprises subjecting the biological sample to a pattern of being pressurized and depressurized for a ramp rate of up to 100 MPa/minute, a hold period at each 100 MPa interval, and then incubated for up to three days at 300 MPa.

26. The system of claim 23, wherein the biological sample comprises a solution having monoclonal antibodies and the predetermined condition includes a pressure cycling profile, wherein the pressure cycling profile comprises subjecting the biological sample to a pressure cycle of being pressurized at a ramp rate of up to 100 MPa/minute and up to 300 MPa, incubated for 30 minutes, depressurized up to 100 MPa/minute and down to 0.1 MPa, and incubated for another 30 minutes, and repeating the pressure cycle ten times over an 11-hour period.

27. (canceled)

28. The system of claim 23, wherein the predetermined condition includes a pressure cycling profile, wherein the pressure cycling profile comprises subjecting the biological sample to a pressure cycle of being pressurized at a ramp rate of up to 10 MPa/second and held at 3 minutes, and wherein the predetermined condition includes the temperature set at 20° C., and repeating the pressure cycle 100 times over an 11-hour period.

29. (canceled)

30. The system of claim 23, wherein the predetermined condition includes a pressure cycling profile, wherein the pressure cycling profile comprises subjecting the biological sample to a pressure cycle of being pressurized at a ramp rate of up to 100 MPa/second and held at 30 seconds.

31. The system of claim 23, wherein the predetermined condition includes a pressure profile, wherein the pressure profile includes subjecting the biological sample to a pattern of being pressurized and depressurized at a ramp rate of 20 MPa/minute until a predetermined pressure is reached, and incubated at 300 MPa for up to three weeks.

32. The system of claim 22, wherein the biological sample comprises a low concentration lysozyme sample (c₂), and the excess Raleigh ratio
$$R \frac{ex}{90 °}$$
can be regressed as a function of concentration according to:
$$\frac{R \frac{ex}{90 °}}{K ( P )} = {M}_{w , app} {c}_{2} + {M}_{2} {G}_{22} {c}_{2}^{2}$$
in order to obtain a Kirkwood-Buff integral (G₂₂) value of net protein-protein interactions (PPIs), wherein M_{w,app} and M₂ are the apparent and true molar masses of the protein, respectively, and wherein optical constant K is defined as
$$K ( P ) = \frac{4 π {n}_{o}^{2}}{{N}_{A} {λ}^{4}} {( \frac{dn}{dc} )}^{2} ,$$
wherein Nₐ represents Avogadro's number, λ represent a wavelength of the laser source, and nₒ is a solvent refractive index, and wherein a relationship between a second virial coefficient (B₂₂) and G₂₂ can be expressed as G₂₂=−2B₂₂.

33. (canceled)

34. An apparatus configured to be secured to a light scattering instrument having a chassis and a laser source configured to emit a laser beam along a path, the apparatus comprising:
a pressure cell mounted to the chassis, the pressure cell comprising a chamber defined by one or more walls and configured to contain a fluid pressurized up to 350 MPa, a plurality of light-transmissive windows in the one or more walls, including at least a beam entry window and a beam exit window positioned in the path of the laser beam, and one or more windows positioned orthogonal to the path of the laser beam, and a sample enclosure positioned in the path of the laser beam, the sample enclosure isolated from, but in temperature and pressure communication with, the fluid;
an external condition-inducing system configured to provide a predetermined temperature and pressure of the fluid within the pressure cell chamber;
a biological sample received in the sample enclosure of the pressure cell; and
a plurality of detectors positioned adjacent one or more of the plurality of windows, the plurality of detectors comprising one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample.

35. A method of for retrofitting an atmospheric light scattering instrument, the atmospheric-pressure light scattering instrument having a chassis and a laser source configured to emit a laser beam along a path, the method comprising:
mounting a pressure cell to the chassis, the pressure cell comprising a chamber defined by one or more walls and configured to contain a fluid pressurized up to 350 MPa, a plurality of light-transmissive windows in the one or more walls, including at least a beam entry window and a beam exit window positioned in the path of the laser beam, and one or more windows positioned orthogonal to the path of the laser beam, and a sample enclosure positioned in the path of the laser beam, the sample enclosure isolated from, but in temperature and pressure communication with, the fluid;
providing an external condition-inducing system configured to provide a predetermined temperature and pressure of the fluid within the pressure cell chamber;
providing one or more openings in the chassis for facilitating a connection between the pressure cell and the external condition-inducing system;
inserting a biological sample into the sample enclosure of the pressure cell;
positioning a plurality of detectors adjacent the pressure cell, the plurality of detectors comprising one or more of a transmission detector disposed adjacent the exit window, and one or more light scattering detectors positioned adjacent the one or more windows orthogonal to the path of the laser beam and configured to detect one or more characteristics of light scattered from the laser beam passing through the sample;
connecting a pressure cable of the external condition-inducing system to the pressure cell via a pressure inlet;
emitting a laser beam along a path toward to the pressure cell, such that when the laser beam enters the sample enclosure of the pressure cell, the light scattered by the biological sample is transmitted through the plurality of windows; and
detecting one or more characteristics of said scattered light by the plurality of detectors.

36. (canceled)

37. (canceled)
